(19) United States
(12) Patent Application Publication
(10) Pub. No.: US 2020/0315620 A1
(43) Pub. Date: Oct. 8, 2020

(54) SURGICAL STAPLER

(71) Applicants: YISI (SUZHOU) MEDICAL TECHNOLOGY CO., LTD., Shanghai (CN); SHANGHAI YISI MEDICAL TECHNOLOGY CO., LTD., Shanghai (CN)

(72) Inventors: Honglin NIE, Shanghai (CN); Guang YANG, Shanghai (CN); Xiliang ZHANG, Shanghai (CN); Xiufeng SHI, Shanghai (CN); Anhua LI, Shanghai (CN); Minghui BAO, Shanghai (CN); Yang JIANG, Shanghai (US)

(21) Appl. No.: 16/303,938

(22) PCT Filed: Jun. 26, 2017
(86) PCT No.: PCT/CN2017/089939

§ 371 Date: Nov. 21, 2018

(30) Foreign Application Priority Data
May 23, 2016 (CN) 201610345074.8

(51) Int. Cl. A61B 17/072

(52) U.S. Cl. CPC A61B 17/072; A61B 2017/0725; A61B 2017/07271

(57) ABSTRACT

The present invention discloses a surgical stapler. The surgical stapler includes a first handle part, a second handle part, and an adjustment mechanism. The first handle part comprises a staple cartridge component and a drive component, and the second handle part comprises a staple abutting seat. A locking mechanism is designed on the first handle part or/and the second handle part. The first handle part and the second handle part may rotate via the rotation shaft and lock in a longitudinal direction. The adjustment mechanism is located between the first handle part and the second handle part, which may make the second handle part rotate relative to the first handle part via the rotation shaft thereon. The surgical stapler provided by the present invention may make, by means of the adjustment mechanism at the proximal end of the handle, the thickness of the tissue after clamping reach the same at the proximal and distal ends, aim to improve the qualification rate of the staples deformation, and reduce the bleeding of the suture orifice, anastomotic leakage and etc.

### TECHNICAL FIELD

The present invention belongs to the technical field of medical instrument and is related to the structure of a stapler, and in particular related to a surgical stapler.

### BACKGROUND

The principle of a surgical stapler is to clamp the tissues by closing the two corresponding handle components (generally including a staple abutting seat and a staple cartridge component), and then push the suturing staple in the cartridge out of shape to suture the tissue together. In some stapler, it is also equipped with a cutting knife for cutting the sutured tissue.

The surgical stapler with the above function includes a first handle part and a second handle part. The first handle part includes a dismountable staple cartridge component, a locking rod, and a drive component. The staple cartridge components generally include a staple cartridge, several suturing staples, and several staple pushing blocks. There are multiple rows of holes on the staple cartridge with suturing staples inside, and the upper surface of the staple cartridge is a tissue contact surface. The second handle part includes a staple abutting seat, with a staple forming surface which contains multiple rows of staple forming grooves.

The first handle part and the second handle are connected and locked by means of the locking rod located on the first handle part to close tissues that need to be sutured.

The drive component includes a wedge pushing sheet. When the drive component moves from the proximal end of the stapler to the distal end, it drives the suturing staple in the staple cartridge to deform in the forming groove of the staple abutting seat. Generally, the drive component also includes a cutting knife used for cutting tissues in the middle of the multiple rows of staples after being sutured.

In an existing stapler design, when the stapler closes the thick tissue, the thickness of the proximal and distal ends of the clamped tissue may be inconsistent after compression due to texture inconsistency of tissues such as toughness, thickness and etc. Generally, the compression thickness generated at distal ends is greater than that of the proximal end, as shown in FIG. 5, and at this time, L2>L1. In this case, the heights of the deformed suturing staples at the proximal and distal ends are inconsistent, which in distal end is greater even with poor qualification of deformation, leading to the bleeding of the suturing orifice, anastomotic leakage and etc.

On such basis, a new stapler is desired to be designed to overcome the above defects of the existing stapler.

### SUMMARY

A technical problem to be solved by the present invention is to provide a surgical stapler which may make the thickness of the tissue after clamping reach the same at the proximal and distal ends, aim to improve the qualification rate of the staples deformation, and reduce the bleeding of the suture orifice, anastomotic leakage and etc.

To solve the above technical problem, the following technical solutions are provided in the present invention:

A surgical stapler, as mentioned, includes a first handle part, a second handle part, and an adjustment mechanism;

The first handle part, as mentioned, includes a staple cartridge base, a dismountable staple cartridge component, a locking rod, and a drive component, and the second handle part includes a staple abutting seat and a rotation shaft;

The first handle part and the second handle part are assembled together, and are locked via the locking rod on the first handle part in a longitudinal direction;

The adjustment mechanism is placed on the proximal end of the stapler and is located between the first handle part and the second handle part; the adjustment mechanism is rotated to make the second handle part rotate relative to the first handle part via the rotation shaft thereon, so that the thickness of the tissue after clamping reach the same at the proximal and distal ends;

The adjustment mechanism is a cam mechanism which contains a rotary dial, a rotary dial knob, a pin, a pull-out piece spring, an adjustment slider, and a sliding pin; the rotary dial is connected to the first handle part via the pin and the pull-out piece spring; the adjustment slider is connected to the second handle part via the sliding pin; a circular hole on the staple cartridge base is coaxially fit to the pin, the rotary dial, and the adjustment slider at the same time;

The rotary dial knob of the adjustment mechanism is connected to the rotary dial which comprises at least two stepped surfaces, and the adjustment slider is engaged with any of the stepped surfaces of the rotary dial. The rotary dial knob rotation drives the rotary dial rotation, leading the adjustment slider engaged with the rotary dial generates displacement vertical to the stepped surface, in which case drive the second handle part via the sliding pin to rotate relative to the first handle part, until the thickness of the tissue after clamping reach the same at the proximal and distal ends.

A surgical stapler, as mentioned, includes a first handle part, a second handle part, and an adjustment mechanism;

The first handle part includes a staple cartridge component and a drive component. The second handle part includes a staple abutting seat. A locking mechanism is designed within the first handle part or/and the second handle part. The first handle part and the second handle part may rotate via a rotation mechanism and lock in a longitudinal direction via the locking mechanism.

The adjustment mechanism is located between the first handle part and the second handle part; the adjustment mechanism is adjusted to make the second handle part rotate relative to the first handle part via the rotation mechanism thereon.

As an optimal solution of the present invention, the adjustment mechanism is disposed on a proximal end of the stapler; the adjustment mechanism adjusts its height so that the second handle part rotates relative to the first handle part, and the thickness of the tissue after clamping by the stapler reach the same at the proximal and distal ends.

As an optimal solution to the present invention, the adjustment mechanism is a cam mechanism.

As an optimal solution of the present invention, the cam mechanism includes a rotary dial knob, a rotary dial, and an adjustment slider. The rotary dial knob is connected to the rotary dial which comprises at least two stepped surfaces. One end of the adjustment slider is engaged with any of the stepped surfaces of the rotary dial, and the other end is connected to the second handle part via a sliding mechanism. The rotary dial knob rotation drives the rotary dial rotation, leading the adjustment slider engaged with the rotary dial generates displacement vertical to the stepped surface, in which case drive the second handle part via the sliding pin to rotate relative to the first handle part, until the thickness of the tissue after clamping reach the same at the proximal and distal ends.

As an optimal solution of the present invention, the adjustment mechanism contains a rotary dial, a rotary dial knob, a pin, a pull-out piece spring, an adjustment slider, and a sliding pin; the rotary dial is connected to the first handle part via the pin and the pull-out piece spring; the adjustment slider is connected to the second handle part via the sliding pin; a circular hole on the staple cartridge base is coaxially fit to the pin, the rotary dial, and the adjustment slider at the same time;

The rotary dial knob is connected to the rotary dial which comprises at least two stepped surfaces. One end of the adjustment slider is engaged with any of the stepped surfaces of the rotary dial, and the other end is connected to the second handle part via a sliding mechanism. The rotary dial knob rotation drives the rotary dial rotation, leading the adjustment slider engaged with the rotary dial generates displacement vertical to the stepped surface, in which case drive the second handle part via the sliding pin to rotate relative to the first handle part, until the thickness of the tissue after clamping reach the same at the proximal and distal ends.

As an optimal solution of the present invention, the adjustment mechanism comprises a wedge slider, which has a sliding slope, a non-horizontal surface. The wedge slider is placed above the staple cartridge base, and the sliding slope fits the staple abutting seat. The sliding slope has a push button which could make the wedge slider locate in different position, leading the staple abutting seat to change the mating surface with the sliding slope, and further making the proximal end of the staple abutting seat raise (or lower) and the distal end lower (or raise), until the thickness of the tissue after clamping reach the same at the proximal and distal ends.

As an optimal solution to the present invention, the adjustment mechanism includes an adjustment screw with thread, the thread of the adjustment screw fits a thread hole located on the first handle part/the second handle part, and the screw top fits the second handle part/the first handle part.

Beneficial effects of the present invention are that: the surgical stapler provided by the present invention may make, by means of the adjustment mechanism at the proximal end of the handle, the thickness of the tissue after clamping reach the same at the proximal and distal ends, aim to improve the qualification rate of the staples deformation, and reduce the bleeding of the suture orifice, anastomotic leakage and etc.

### ILLUSTRATION OF ATTACHED FIGURES

FIG. 1 is a schematic structural diagram of a surgical stapler of the present invention in embodiment 1;

FIG. 2 is a schematic structural diagram of an adjustment mechanism in the surgical stapler of the present invention in embodiment 1;

FIG. 3 is a schematic structural diagram of a rotary dial in the surgical stapler of the present invention in embodiment 1;

FIG. 4 is a schematic structural diagram of an adjustment slider in the surgical stapler of the present invention in embodiment 1;

FIG. 5 is a schematic diagram of the surgical stapler before adjustment in embodiment 1;

FIG. 6 is a schematic diagram of the surgical stapler after adjustment in embodiment 1;

FIG. 7 is a schematic diagram of an adjustment mechanism in the surgical stapler of the present invention in embodiment 2;

FIG. 8 is a schematic diagram of a wedge slider in the surgical stapler of the present invention in embodiment 2;

FIG. 9 is a schematic diagram of a surgical stapler of the present invention in embodiment 3; and

FIG. 10 is a schematic diagram of an adjustment screw in the surgical stapler of the present invention in embodiment 3.

### EMBODIMENTS

Preferred embodiments of the present invention are described in detail in combination with accompany drawings.

### Embodiment 1

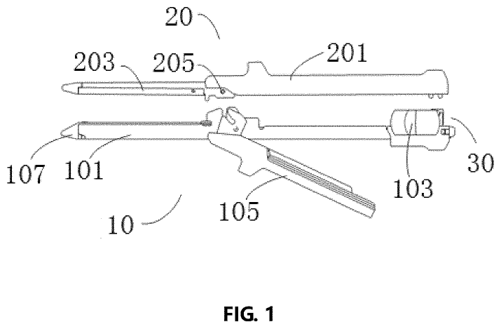

Referring to FIGS. 1 to 6, the invention discloses a surgical stapler, and the surgical stapler includes a first handle part 10, a second handle part 20, and an adjustment mechanism 30.

In this embodiment, the first handle part 10 includes a staple cartridge base 101, a dismountable staple cartridge component (including a staple cartridge 107, several staple pushing blocks, and several suturing staples, a locking rod 105, a drive component (a pushing button 103, a pushing sheet, and a cutting knife in this embodiment); the locking rod 105 is rotatably installed on the first handle part 10. The second handle part 20 includes a staple abutting seat housing 201, a staple abutting seat 203, and a rotation shaft 205 (or other rotation mechanisms), and the staple abutting seat 203 has a staple forming surface.

The first handle part 10 and the second handle part 20 are assembled together, and are locked via the locking rod 105 on the first handle part 10 in a longitudinal direction.

The adjustment mechanism 30 is placed on a proximal end of the stapler and is located between the first handle part 10 and the second handle part 20 (more specifically, between the staple cartridge base 101 and the staple abutting seat 203); the adjustment mechanism 30 is rotated to make the second handle part 20 rotate relative to the first handle part 10 via the rotation shaft 205 thereon, so that the thickness of the tissue after clamping reach the same at the proximal and distal ends.

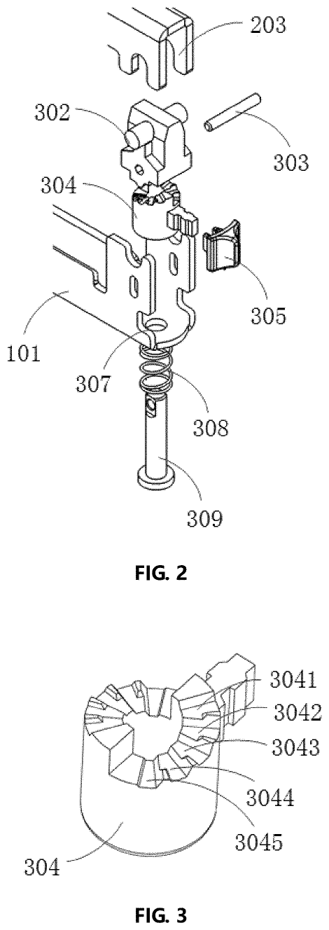

Referring to FIG. 2, in this embodiment, the adjustment mechanism 30 is a cam mechanism. The cam mechanism includes a rotary dial knob 305, a rotary dial 304, a pin 309, a pull-out piece spring 308, an adjustment slider 302, a sliding pin 303 (or any other sliding mechanism).

The rotary dial 304 is connected to the first handle part 10 via the pin 309 and the pull-out piece spring 308; the adjustment slider 302 is connected to the second handle part 20 via the sliding pin 303; a circular hole is arranged on the staple cartridge base 101, and is coaxially fit to the pin 309, the rotary dial 304, and the adjustment slider 302, as shown in FIG. 2.

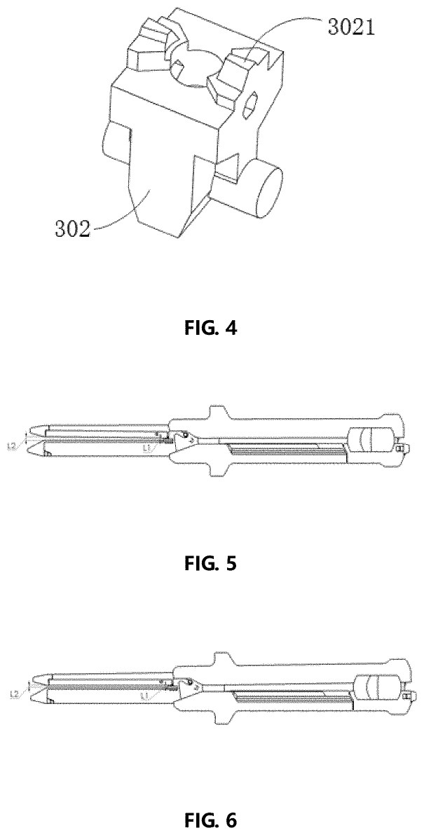

As shown in FIG. 3 and FIG. 4, the rotary dial knob 305 is connected to the rotary dial 304, and the rotary dial 304 includes at least two stepped surfaces 3041, 3042, 3043, 3044, and 3045 (as shown in FIG. 3). One end of the adjustment slider 302 is engaged with any of the stepped surface of the rotary dial 304, and the other end is connected to the sliding pin 303 by means of the second handle part 20. When the rotary dial button 305 is rotated, the rotary dial 304 also rotates, so that the adjustment slider 302 (including a second stepped surface 3021 fitting the stepped surface of the rotary dial 304) engaged with the rotary dial generates displacement vertical to the stepped surface, so as to drive the second handle part 20 via the sliding pin 303 to rotate relative to the first handle part 10, until the thickness of the tissue after clamping reach the same at the proximal and distal ends, as shown in FIG. 6, and at this time, L1≈L2.

Certainly, the adjustment mechanism may use other methods to make the second handle part rotate relative to the first handle part, so that the thickness of the tissue after clamping reach the same at the proximal and distal ends.

### Embodiment 2

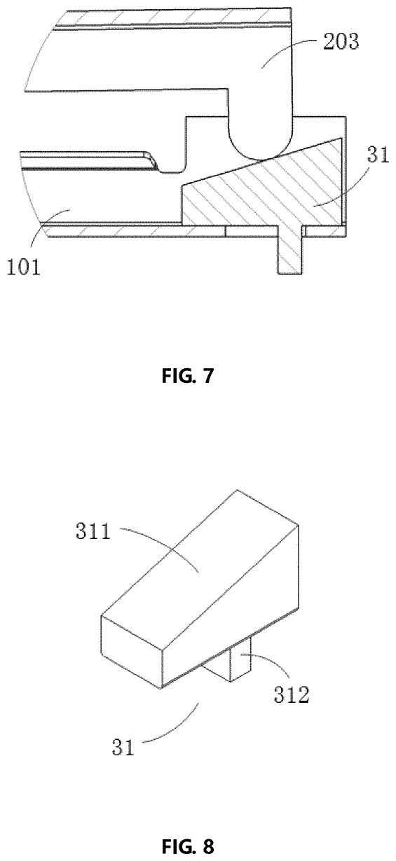

This embodiment differs from embodiment 1 in that in this embodiment, referring to FIGS. 7 and 8, the adjustment mechanism includes a wedge slider 31, which has a sliding slope 311, and the sliding slope 311 is a non-horizontal surface. The wedge slider 31 is placed above the staple cartridge base 101, and the sliding slope 311 fits the staple abutting seat 203. The sliding slope 311 has a push button 312. The push button 312 may make the wedge slider 31 locate in different position, so that the staple abutting seat 203 changes the mating surface with the sliding slope 311, and further make the proximal end of the staple abutting seat 203 raise (or lower) and the distal end lower (or raise), until the thickness of the tissue after clamping reach the same at the proximal and distal ends, as shown in FIG. 6, at this time, L1≈L2.

### Embodiment 3

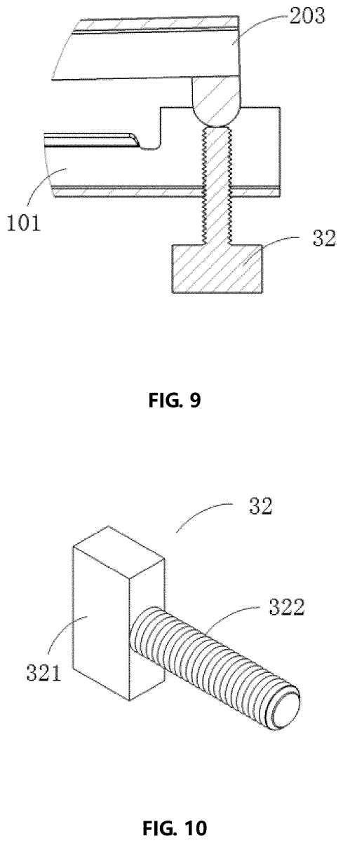

This embodiment differs from embodiment 1 in that in this embodiment, referring to FIGS. 9 and 10, the adjustment mechanism includes an adjustment screw with thread 32 (including an adjustment knob 321 and the thread 322), the thread of the adjustment screw 32 fits the screw hole located on the staple cartridge base 101, and the screw top fits the staple abutting seat 203.

When the adjustment knob on the adjustment screw is rotated manually, the relative position of the adjustment screw top to the staple cartridge base changes, so that the proximal end of the staple abutting seat 203 raise (or lower) and the distal end lower (or raise), until the thickness of the tissue after clamping reach the same at the proximal and distal ends, as shown in FIG. 6, L1≈L2.

### Embodiment 4

A surgical stapler, includes a first handle part, a second handle part, and an adjustment mechanism. The first handle part includes a staple cartridge component and a drive component. The second handle part includes a staple abutting seat; a locking mechanism is disposed on the first handle part or/and the second handle part, and the first handle part and the second handle part may rotate via the rotation shaft (or any other rotation mechanism) and is locked via the locking mechanism in a longitudinal direction. The adjustment mechanism is located between the first handle part and the second handle part. The second handle part can rotate relative to the first handle part, via the rotation shaft thereon.

In conclusion, the surgical stapler as described above may make, by means of the adjustment mechanism at the proximal end of the handle, the thickness of the tissue after clamping reach the same at the proximal and distal ends, aim to improve the qualification rate of the staples deformation, and reduce the bleeding of the suture orifice, anastomotic leakage and etc.

The description and application of the invention are intended to be illustrative, not only limited to the scope of above. Variations and modifications of the embodiments disclosed herein are possible. Replacements and various equivalent alternatives of the components are well known to those skilled personnel in the art. It is apparent to those skilled in the art that the present invention may be embodied in other forms, structures, arrangements, ratios, and other assemblies, materials and components without deviating from the spirit or essential characteristics of the invention. Other variations and modifications of the embodiments disclosed herein may be made without deviating from the scope and spirit of the invention.

1. (canceled)

2. A surgical stapler comprising:
a first handle part including a staple cartridge component and a drive component;
a second handle part including a staple abutting seat;
a locking mechanism arranged on one of the first handle part and the second handle part, wherein the first handle part and the second handle part rotate via a rotation mechanism and lock in a longitudinal direction via the locking mechanism; and
an adjustment mechanism located between the first handle part and the second handle part and configured to rotate the second handle part relative to the first handle part via the rotation mechanism thereon.

3. The surgical stapler according to claim 2, wherein
the adjustment mechanism is located on a proximal end of the stapler and configured to adjust its height so that the second handle part rotates relative to the first handle part, and a thickness of a tissue after being clamped by the stapler is same at the proximal end and a distal end.

4. The surgical stapler according to claim 2, wherein the adjustment mechanism is a cam mechanism.

5. The surgical stapler according to claim 4, wherein
the cam mechanism includes a rotary dial knob, a rotary dial, and an adjustment slider.

6. The surgical stapler according to claim 2, wherein
the adjustment mechanism includes a rotary dial, a rotary dial knob, a pin, a pull-out piece spring, an adjustment slider, and a sliding pin, and wherein the rotary dial is connected to the first handle part via the pin and the pull-out piece spring, and the adjustment slider is connected to the second handle part via the sliding pin.

7. The surgical stapler according to claim 2, wherein
the adjustment mechanism comprises a wedge slider having a sliding slope, the wedge slider being disposed above the staple cartridge base, and wherein the sliding slope fits the staple abutting seat.

8. The surgical stapler according to claim 2, wherein the adjustment mechanism comprises an adjustment screw with a thread and a screw top, the thread of the adjustment screw fits in a screw hole located on the first handle part, and the screw top fits in the second handle part.

9. The surgical stapler according to claim 5, wherein the rotary dial knob is connected to the rotary dial and includes at least two stepped surfaces.

10. The surgical stapler according to claim 9, wherein one end of the adjustment slider is engaged with any one of the stepped surfaces of the rotary dial, and another end of the adjustment slider is connected to the second handle part via a sliding mechanism.

11. The surgical stapler according to claim 10, wherein a rotation of the rotary dial knob drives the rotary dial along with the knob causing the adjustment slider to generate a vertical displacement with respect to the stepped surface.

12. The surgical stapler according to claim 11, wherein the rotation of the rotary dial knob causes the second handle part via the sliding mechanism to rotate relative to the first handle part until a thickness of the tissue after being clamped by the stapler is same at a proximal end and a distal end.

13. The surgical stapler according to claim 6, wherein the staple cartridge base includes a circular hole that is coaxial with respect to the pin, the rotary dial, and the adjustment slider.

14. The surgical stapler according to claim 7, wherein the wedge slider includes a push button, which upon being pressed causes a displacement of the staple abutting seat along the sliding slope the wedge slider.

15. The surgical stapler according to claim 14, wherein upon the push button being pressed, a proximal end of the staple abutting seat is raised/lowered, and a distal end is lowered/raised until a thickness of a tissue after being clamped is same at the proximal end and the distal end.
